(19) United States
(12) Patent Application Publication
(10) Pub. No.: US 2008/0149512 A1
(43) Pub. Date: Jun. 26, 2008

(54) MEDICAL INSTRUMENT BRACKET ASSEMBLY

(76) Inventor: Gary T. Dane, Bow, NH (US)

Correspondence Address: HAYES, SOLOWAY P.C., 175 CANAL STREET, MANCHESTER, NH, 03101, US

(21) Appl. No.: 11/963,202

(22) Filed: Dec. 21, 2007

Related U.S. Application Data
(60) Provisional application No. 60/871,644, filed on Dec. 22, 2006.

(51) Int. Cl. A61B 19/00; B65D 1/34

(52) U.S. Cl. 206/370

(57) ABSTRACT

A bracket assembly for supporting medical instruments and a method of producing the bracket assembly are provided. Generally the bracket assembly contains a lower portion made of a resilient material adapted to attach to a tray and an upper portion made of a flexible material adapted to receive a medical instrument.

### CROSS-REFERENCES

This application claims priority to co-pending U.S. Provisional Application entitled, “Medical Instrument Bracket Assembly,” having Ser. No. 60/871,644, filed Dec. 22, 2006, which is incorporated herein by reference.

### FIELD OF THE INVENTION

The present invention is generally related to a bracket assembly, and more particularly is related to a bracket assembly with a lower resilient portion and an upper flexible portion.

### BACKGROUND OF THE INVENTION

Surgical instruments are often held in trays prior to and during use. The instruments are usually laid out in a certain way in the tray so that they can be picked from the tray in the order required for a particular procedure. Instrument supporting brackets can be used to hold the instruments and can take several different forms.

Currently, brackets may be made of a variety of materials such as rigid plastics, silicone, stainless steel and aluminum. However, one problem with current brackets is that the medical instruments need to be rigidly attached to a tray to keep the instrument and/or bracket from falling loose during sterilization or transport. The brackets also need to be flexible enough to receive hard instruments of various sizes. Another problem with the current bracket system is that bracket particulates may chip off of the bracket and attach to the instrument. These particulates may be dangerous to a patient if inserted into a patient during a surgical procedure.

Thus, a heretofore unaddressed need exists in the industry to address the aforementioned deficiencies and inadequacies.

### SUMMARY OF THE INVENTION

Embodiments of the present invention provide a bracket and method for forming the bracket for use on a medical tray. Briefly described, in architecture, one embodiment of the device, among others, can be implemented as follows. The device contains a lower portion made of a resilient material adapted to attach to each tray and an upper portion made of a flexible material adapted to receive an instrument.

The present invention can also be viewed as providing methods for forming a bracket with a lower portion made of a resilient material adapted to attach to a tray and an upper portion made of a flexible material adapted to receive an instrument. In this regard, one embodiment of such a method, among others, can be broadly summarized by the following steps: extruding a piece of resilient material, extruding a portion of flexible material, combining the resilient material and the flexible material to form a bracket, attaching a bracket to a medical tray.

Other devices, systems, methods, features, and advantages of the present invention will be or become apparent to one with skill in the art upon examination of the following drawings and detailed description. It is intended that all such additional systems, methods, features, and advantages be included within this description, be within the scope of the present invention, and be protected by the accompanying claims.

### BRIEF DESCRIPTION OF THE DRAWINGS

Many aspects of the invention can be better understood with reference to the following drawings. The components in the drawings are not necessarily to scale, emphasis instead being placed upon clearly illustrating the principles of the present invention. Moreover, in the drawings, like reference numerals designate corresponding parts throughout the several views.

FIG. 1 is a perspective view illustrating a bracket in accordance with a first exemplary embodiment of the invention

FIG. 2 is a perspective view illustrating another bracket in accordance with the first exemplary embodiment of the invention.

FIG. 3 is a top view of a tray with a plurality of brackets in accordance with the first exemplary embodiment of the present invention.

FIG. 4 is a side view illustrating a tray with a bracket in accordance with the first exemplary embodiment of the present invention.

FIG. 5 is a flow chart illustrating a method for providing a bracket assembly in accordance with the first exemplary embodiment of the invention.

### DETAILED DESCRIPTION

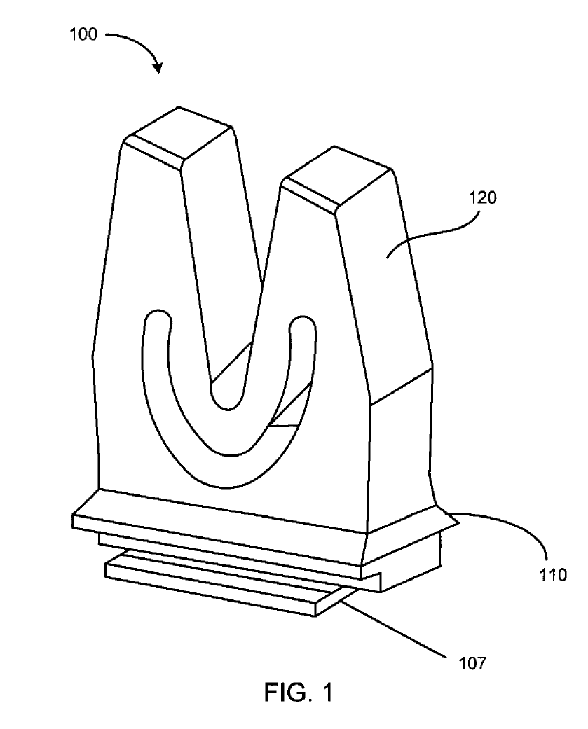

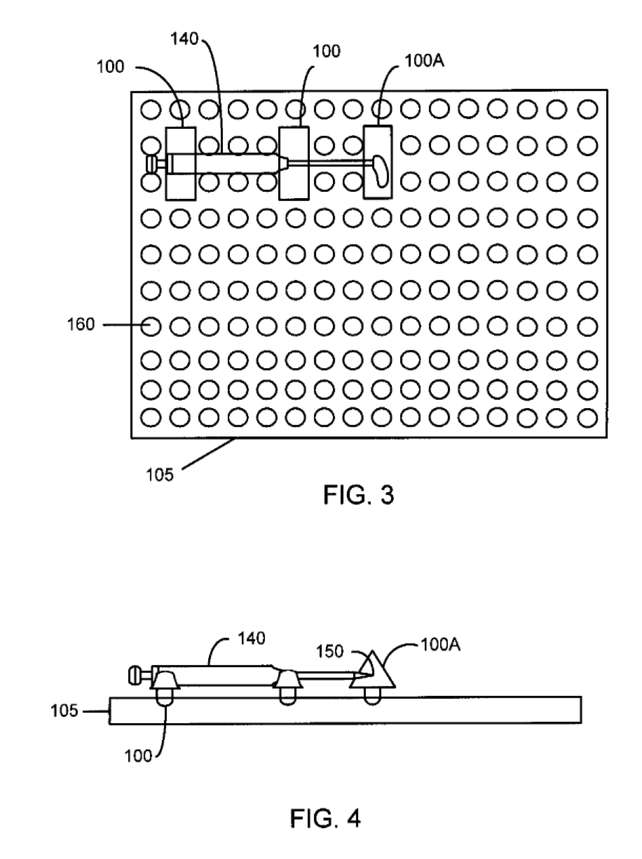

FIG. 1 is a perspective view illustrating a bracket 100 in accordance with a first exemplary embodiment of the invention. The bracket 100 contains a lower portion 110 made of a resilient material adapted to attach to a medical tray 105 (shown in FIG. 3) and an upper portion 120 made of a flexible material adapted to receive a medical instrument 140 (shown in FIG. 3). The lower portion 110 and the upper portion 120, in accordance with the first exemplary embodiment, may each be made of silicone of different durometers. More specifically, the lower portion 110 may be made of a high durometer silicone, such as a silicone having a durometer of at least approximately 70 on a shore A scale. The upper portion 120 may be made of a low durometer silicone, such as a durometer approximately between one third and two thirds of the durometer for the lower portion 110. For instance, the upper portion 120 may be a 35 durometer silicone and the low portion 110 may be a 70 durometer silicone.

Silicones, known chemically as polyorganosiloxanes, have a backbone consisting of alternating silicon and oxygen atoms, which allows the backbone to be modified by attaching different organic groups to the backbone. Nearly all silicone products are derived from the following three types of raw materials: silicone fluids, silicone rubbers and silicone resins.

Silicone fluids are linear polymers whose chains contain between 2 and well over 1,000 atoms, each of which is linked to the next by an oxygen atom. Silicone fluids change very little in viscosity over a wide temperature range and are thus beneficial for forming brackets for medical instruments that require sterilization. Silicone rubbers are long-chain polysiloxanes, that are converted to an elastomeric (rubbery) state by vulcanization. Silicone resins range from relatively low-molecular intermediates to high-molecular densely crosslinked resins of a wide variety of structures. Silicone resins typically are highly resistant to heat.

In accordance with the first exemplary embodiment of the present invention the lower portion 110 may be made of silicone rubber. In this embodiment, the silicone rubber is vulcanized to produce a resilient material with high durometer for sturdy attachment of the bracket 100 to the tray 105 (shown in FIG. 3). The upper portion 120, in accordance with the present embodiment, is also made of silicone rubber that is vulcanized to produce a flexible material with a low durometer that is adapted for receiving an instrument. The lower portion 110 and the upper portion 120 may be co-extruded. Co-extrusion involves extruding the parts separately, but joining them together as they come out of extrusion.

The bracket 100 may attach directly to the tray 105 (shown in FIG. 3), for instance, by inserting a screw up through the tray 105 and into the lower portion 110. The bracket 100 may attach indirectly to the tray 105 (shown in FIG. 3), for instance, by connecting the bracket to a stand that is mechanically fastened to the tray 105. As shown in FIG. 1, a base 107 of the bracket 100 may be formed to slide into and friction fit with a stand. Those having ordinary skill in the art will recognize there are other structures for attaching a bracket to a tray and those structures are considered to be within the scope of the present invention.

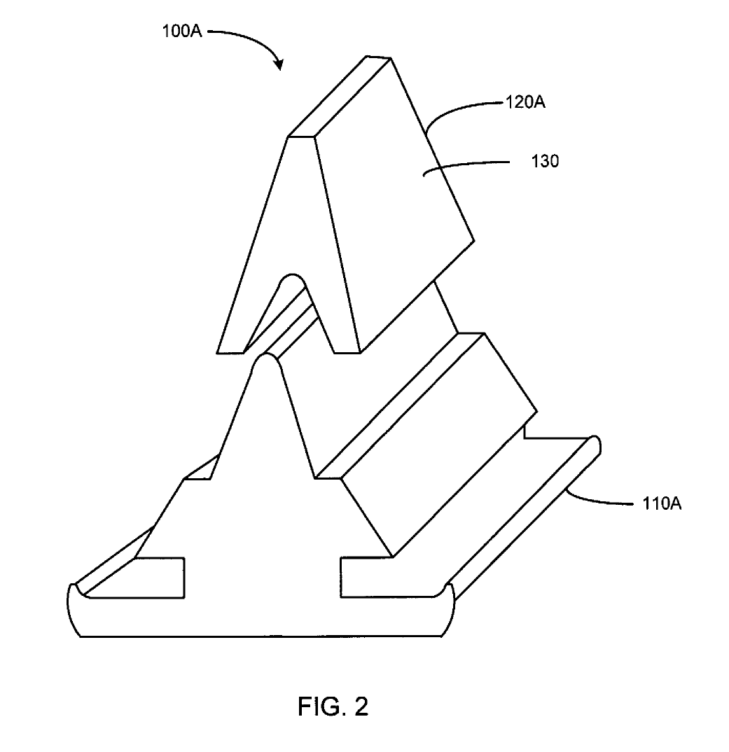

FIG. 2 is a perspective view illustrating another bracket 100A in accordance with the first exemplary embodiment of the invention. The bracket 100A has a stopper portion 130 forming the upper portion 120A. The stopper portion 130 is made of a flexible material with a low durometer silicone that is adapted for receiving an instrument. The stopper portion 130 acts as a receiver for the blunt edge 150 of a medical instrument 140, as shown in FIG. 4. The lower portion 110A is made of a high durometer silicone for sturdy attachment of the bracket 100A to a tray 105 (shown in FIG. 3).

FIG. 3 is a top view of the tray 105 with a plurality of brackets 100, 100A in accordance with the first exemplary embodiment of the present invention. The tray 105 may be an aluminum tray or a plastic sterilization tray or other material acceptable for use in a tray for holding medical instruments in a surgical procedure. A plurality of apertures 160 are shown in the tray 105 for receiving brackets 100, 100A. The brackets 100, 100A may have extensions (not shown) that attach to the plurality of apertures 160. The brackets 100, 100A may fit to stands that are mechanically fastened to the tray 105. The brackets 100, 100A may receive screws that are inserted from beneath the tray 105 and through the apertures 160. The lower portion 110, 110A of the brackets 100, 100A may have preformed holes for receiving screws to attach the brackets 100, 100A to the tray 105.

The stopper portion 130 of the second bracket 100A receives the blunt edge 150 of medical instrument 140. The stopper portion 130 may prevent a surgeon or other hospital personnel from cutting or injuring themselves when reaching for medical instruments 140 on tray 105. In this embodiment, the stopper portion 130 may be made of silicone rubber that is vulcanized to produce a flexible material with a low durometer that is adapted for receiving an instrument. However, those having ordinary skill in the art will recognize the stopper portion 130 may be made of a silicone resin or silicone fluids to produce a flexible material, which is considered to be within the scope of the present invention.

FIG. 4 is a side view illustrating the tray 105 shown in FIG. 3, in accordance with the first exemplary embodiment of the present invention. The bracket 100A is shown holding the medical instrument 140. In this embodiment, a blunt edge 150 is shown penetrating the stopper portion 130 of the bracket 100A such that a user does not get hurt while reaching for and handling medical instruments 140.

There are several methods to manufacture brackets 100, 100a in accordance with the present invention. Some of these methods include extrusion, casting, compression molding, injection molding, blow molding, pressure forming, bonding, adhesive bonding, thermoforming, chemical machining, electrical machining, liquid resin molding, reaction injection molding (RIM), rotational molding, resin transfer molding (RTM), powder metallurgy and any other methods that are understood by those skilled in the art. For the purposes of the embodiments described herein, an extrusion process will be described for forming brackets 100, 100A.

Extrusion is a process by which many types of parts can be produced. The cross-sections that can be produced vary from solid round, rectangular, to L shapes, and T shapes. Extrusion is accomplished in accordance with the embodiments of the present invention by squeezing silicone in a closed cavity through a tool, known as a die using either a mechanical or hydraulic press. The brackets 100, 100A may, for example, be produced using either hot extrusion or cold extrusion.

Cold extrusion is a process that is done at room temperature or slightly elevated temperatures. This process can be used for most materials-subject to designing, such as silicones, that are robust enough to withstand the stresses created by extrusion. In the embodiments described, the lower portion 110, 110A and the upper portion 120, 120a can be extruded using a cold extrusion method to produce the bracket 100, 100A wherein the lower portion 110, 110A and the upper portion 120, 120A are securely formed into a single bracket 100, 100A using an adhesive or mechanically fixing the two portions. Those having ordinary skill in the art will recognize there are various known methods of attaching dissimilar materials and those methods are considered to be within the scope of the present invention.

Hot extrusion is a process done at fairly high temperatures, approximately 50 to 75% of the melting point of the silicone. The pressures can range from 35-700 MPa (5076-101,525 psi). In the embodiments described, the lower portion 110, 110A and the upper portion 120, 120A can be extruded using a hot extrusion method to produce the bracket 100, 100A wherein the lower portion 110, 110A and the upper portion 120, 120A are securely formed into the bracket 100, 100A. Co-extrusion may be more amenable to hot extrusion, after which the extruded materials remain tacky and are more easily joined.

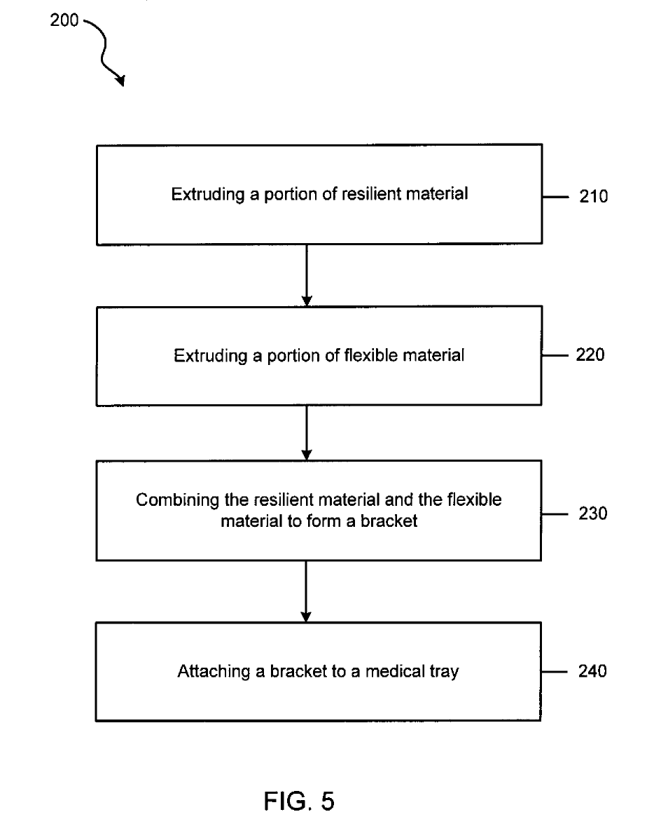

FIG. 5 is a flow chart 200 illustrating a method for providing a bracket assembly 100, 100A in accordance with the first exemplary embodiment of the invention. It should be noted that any process descriptions or blocks in flow charts should be understood as representing modules, segments, portions of code, or steps that include one or more instructions for implementing specific logical functions in the process, and alternate implementations are included within the scope of the present invention in which functions may be executed out of order from that shown or discussed, including substantially concurrently or in reverse order, depending on the functionality involved, as would be understood by those reasonably skilled in the art of the present invention.

A piece of resilient material is extruded (block 210). A portion of flexible material is extruded (block 220). The resilient material and the flexible material are combined to form a bracket 100A (block 230). The bracket 100a attaches to a medical tray 105 (block 240).

It should be emphasized that the above-described embodiments of the present invention, particularly, any “preferred” embodiments, are merely possible examples of implementations, merely set forth for a clear understanding of the principles of the invention. Many variations and modifications may be made to the above-described embodiments of the invention without departing substantially from the spirit and principles of the invention. All such modifications and variations are intended to be included herein within the scope of this disclosure and the present invention and protected by the following claims.

What is claimed is:

1. A bracket assembly for supporting medical instruments, said bracket assembly comprising:
a tray;
a lower portion made of a resilient material adapted to removably attach to the tray; and
an upper portion made of a flexible material adapted to receive a medical instrument, the upper portion attached to the lower portion.

2. The bracket assembly of claim 1, wherein the resilient material is a silicone material.

3. The bracket assembly of claim 1 wherein the resilient material is a high durometer material.

4. The bracket assembly of claim 1 wherein the flexible material is a silicone material.

5. The bracket assembly of claim 1 wherein the flexible material is a low durometer material.

6. The bracket assembly of claim 1, wherein the tray is a sterilization tray adapted to support medical instruments for sterilization.

7. The bracket assembly of claim 1, wherein the upper portion and the lower portion are mechanically interlocked.

8. The bracket assembly of claim 1, where wherein the upper portion and the lower portion are adhesively joined.

9. A method of providing a bracket assembly, comprising the steps of:
extruding a piece of resilient material,
extruding a portion of flexible material,
combining the resilient material and the flexible material to form a bracket,
attaching the bracket to a medical tray.

10. The method of claim 9, further comprising permanently attaching the bracket to the medical tray.

11. The method of claim 9, further comprising mechanically interlocking the resilient material and the flexible material.

12. The method of claim 9, further comprising adhesively joining the resilient material and the flexible material.

13. The method of claim 9, wherein the resilient material has a materially higher durometer than the flexible material.

14. The method of claim 9, wherein the resilient material and the flexible material are co-extruded.

15. A bracket assembly for supporting medical instruments, said bracket assembly comprising:
a tray;
a lower portion made of a resilient material having a durometer of approximately at least 85, the lower portion adapted to removably attach to the tray; and
an upper portion made of a flexible material having a durometer of approximately between 35 and 55, the upper portion adapted to receive a medical instrument, wherein the upper portion attached to the lower portion.

16. The bracket assembly of claim 15, wherein the lower portion and the upper portion are co-extruded.

17. The bracket assembly of claim 15, wherein the lower portion is attached to the tray with at least one screw inserted through holes in the tray from an underside of the tray.

18. The bracket assembly of claim 17, wherein holes are preformed in the lower portion to receive the screws.

19. The bracket assembly of claim 15, wherein the lower portion and the upper portion are formed of silicone.
